(19) United States
(12) Patent Application Publication
(10) Pub. No.: US 2006/0211975 A1
(43) Pub. Date: Sep. 21, 2006

(54) Male genitalia support device, and method for supporting male genitalia

(76) Inventor: Wm. Michael Mular, Bartlett, TN (US)

Correspondence Address: PETER L. BREWER BEARMAN, CALDWELL, & BERKOWITZ;LAW FIRM, 165 MADISON AVENUE, SUITE 2000, MEMPHIS, TN, 38103, US

(21) Appl. No.: 11/080,168

(22) Filed: Mar. 15, 2005

(51) Int. Cl. A61F 13/00

(52) U.S. Cl. 602/70

(57) ABSTRACT

A device for supporting male genitalia of a patient in a recumbent position is provided. The device generally includes a scrotal support member having first and second opposite ends, at least one lateral support strap secured to each of the first and second ends, and a plurality of pads corresponding to a respective lateral support strap, each pad having a first surface to be releasably secured to the patient, and a second surface for receiving the respective lateral support strap. The pad preferably releasably receives a corresponding support strap. A method for supporting male genitalia using a support device is also provided.

### BACKGROUND OF THE INVENTION

1. Field of the Invention

The present invention relates to medical devices for males. More specifically, the invention relates to a device for supporting male genitalia, and a method of supporting male genitalia.

2. Description of the Related Art

In order to provide support for male genitalia, most men wear a supporting undershort. Such undershorts, sometimes referred to as “briefs,” support the genitalia during everyday activities of standing, sitting, walking, running and exercising.

When an older male becomes bedridden either due to age or infirmity, it is unnecessary for him to wear briefs. Indeed, briefs may interfere with the ability of health care providers such as nurses to assist a male patient in keeping the genitalia clean, in monitoring the progress of surgical procedures related to a male organ, or in performing bodily functions. At the same time, a need for some male organ support remains.

The scrotum is a sensitive part of the male anatomy. In some older men the scrotum can become heavy and can hang down between a patient's legs and touch the bedsheets. The scrotum has thin skin, and contact with the scrotum can be painful. In addition, in patients that have limited movement, feces and fluids may collect around the scrotum and irritate the skin. Further, in some ill patients the scrotum itself may collect fluids and become swollen. The skin then begins to break down and may become infected.

U.S. Pat. No. 6,308,710, issued to Silva, presents a “Scrotal Drape and Support.” The drape is generally “V”-shaped and functions as a sling to hold the scrotum upwardly. The scrotal device is designed to be used during medical analysis and treatment. Such a device was not developed to maintain the scrotum in a natural and comfortable position when the male patient is resting or sleeping.

There is a need, therefore, for a medical device that elevates the scrotum to keep it from hanging down between the thighs of a male patient in a recumbent position. Further, there is a need for a support device for male genitalia that may be positioned to support the patient while the patient is lying down in a bed or sitting in a reclined chair or bed. Still further, there is a need for a method for supporting male genitalia that does not require the placement of briefs or support device around the patient's waist. In addition, there is a need for a soft, light-weight and disposable male support device.

### SUMMARY OF THE INVENTION

A device for supporting male genitalia of a patient in a recumbent position is provided. In one embodiment, the device includes a scrotal support member having first and second opposite ends; at least one lateral support strap secured to each of the first and second ends; and a plurality of pads corresponding to a respective lateral support strap, each pad having a first surface to be releasably secured to the patient, and a second surface for releasably receiving the respective lateral support strap. The scrotal support member may have one, two or more lateral support straps on each side.

Preferably, each of the plurality of pads has an adhesive material on the first surface, and an attachment member on the second surface for connecting to a corresponding attachment feature on the respective lateral support strap. The attachment arrangement may be by hook-and-loop, by snaps, or by other connector arrangement.

A method for supporting male genitalia of a patient in a recumbent position is also provided. In one aspect, the method includes the steps of securing a first skin pad on the patient on one side of his body; securing a second skin pad on the patient on the other side of his body; providing a scrotal support member, the scrotal support member having first and second opposite ends with a lateral support strap secured to each of the first and second ends; placing the scrotal support member under the patient's genitalia; and securing each of the lateral support straps to a corresponding skin pad so as to support the genitalia of the patient. The scrotal support member may again have one, two or more lateral support straps on each side.

In one aspect, a first pair of skin pads is placed below the patient's waistline. In another aspect, a second pair of skin pads is placed along the patient's upper thighs. The skin pads preferably have a first adhesive surface to be releasably secured to the patient, and a second surface for releasably receiving a corresponding lateral support strap. In one aspect, the method further comprises securing a catheter to the scrotal support member.

### BRIEF DESCRIPTION OF THE DRAWINGS

So that the manner in which the above recited features of the present invention can be better understood, certain drawings or flow charts are appended hereto. It is to be noted, however, that the appended drawings illustrate only selected embodiments of the inventions and are therefore not to be considered limiting of scope, for the inventions may admit to other equally effective embodiments and applications.

FIG. 1 presents a perspective view of the male organ support device, in one embodiment. Visible in this view is a scrotal support member along with two pairs of lateral support straps. Each strap is shown exploded away from a corresponding skin pad. The straps are releasably securable to the skin pads.

FIG. 2A shows a bottom view of a skin pad, in one embodiment. In this embodiment, the skin pad defines an annular ring. An adhesive material is placed on the bottom surface of the skin pad.

FIG. 2B is a top view of the skin pad of FIG. 2A.

FIG. 3 is a perspective view of a male support device, in an alternate embodiment.

### DETAILED DESCRIPTION

Definitions

As used herein, the term “patient” refers to any male in need of a support device for genitalia while in the recumbent position, including by way of example only a hospital patient, a surgical patient in a surgical facility, or a resident of a nursing home or extended care facility.

The term “recumbent position” is not limited to the means by which the patient is recumbent. The patient may be on a bed, on a chair, on a gurney, on a cot, in a medical vehicle or other apparatus for supporting the patient.

### Description of Specific Embodiments

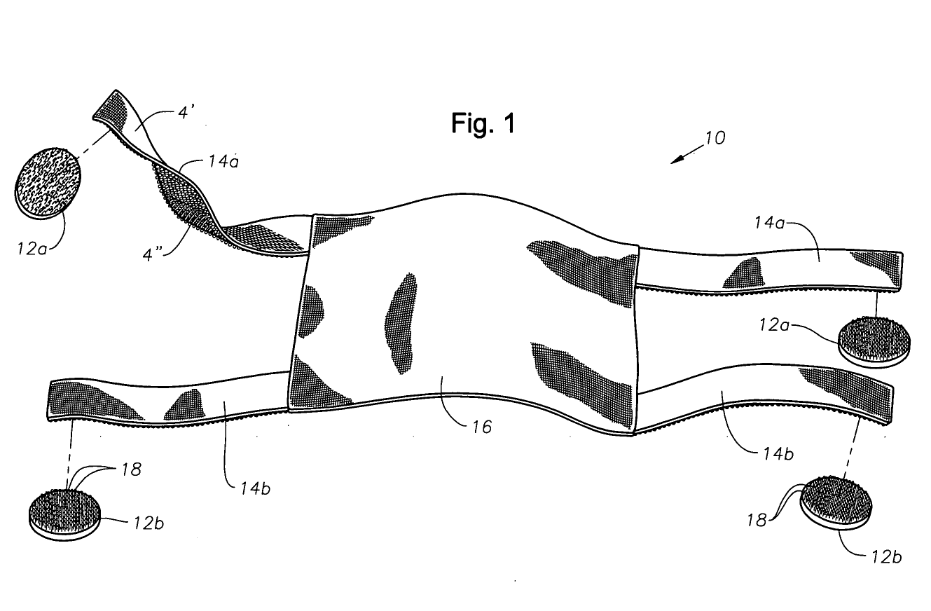

FIG. 1 presents a perspective view of a male organ support device 10, in one embodiment. Visible in this view is a scrotal support member 16. The scrotal support member 16 is preferably fabricated from a soft and light-weight material. Further, the scrotal support member 16 is preferably fabricated from a breathable, absorbent material such as cotton or a cotton-poly blend. The scrotal support member 16 may optionally have an outer plastic lining to prevent moisture from contacting the support member 16 from underneath the patient.

The male organ support device 10 also includes lateral support straps 14. In the embodiment of FIG. 1, two pairs of straps are shown. One pair is referenced as 14a, while the other pair is referenced as 14b. However, it is understood that a single pair of straps 14 may be used, or more than two straps 14 may be provided.

Each strap 14 has a first end and a second end. A first end of each strap 14 is affixed to the scrotal support member 16. The second opposite end is a free end that is designed to be releasably connectible to a skin patch, or pad. Corresponding pads are shown at 12. Pads 12a correspond to straps 14a, while pads 12b correspond to straps 14b. Each strap 14 is shown in FIG. 1 exploded away from a corresponding skin pad 12.

In order to connect a strap 14a, 14b to a corresponding pad 12a, 12b, a connector is provided. Preferably, the connector is a hook and loop arrangement. In this arrangement, each strap 14a, 14b has a top side 4′ and a bottom side 4″. At least a portion of the bottom side 4″ would be fabricated from a loop material; while at least a portion of the corresponding patch 12 is fabricated from a material having hooks. Those of ordinary skill in the art will understand from this disclosure that the loop material may alternatively be placed on the patch 12, while the hook material may then be disposed on the strap 14 proximal to the free end of the strap 14. It is further understood that other connector arrangements may be employed, such as the use of metal snaps or adhesive. However, the hook-and-loop arrangement is preferred as it allows the straps 14 to be conveniently connected and disconnected, and to be longitudinally adjusted to best fit the patient.

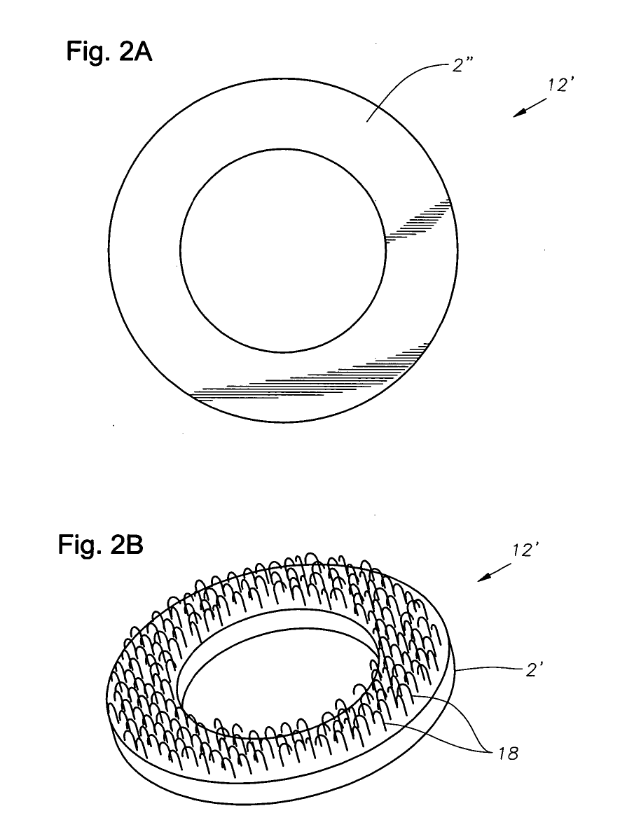

In the view of FIG. 1, the individual pads 12 are generally solid and circular. However, other shapes may be utilized. FIG. 2A shows a bottom view of a skin pad 12′, in an alternate embodiment. In this embodiment, the skin pad 12′ defines an annular ring with an opening therein. An adhesive material is placed on a bottom surface 2″ of the skin pad 12′. Where a solid pad is used, the adhesive point on the reverse side need not cover the entire back surface; various sizes for the adhesive attachment point may be utilized.

FIG. 2B is a top view of the skin pad 12′ of FIG. 2A. A roughened material is shown on a top surface 2′ of the annular ring. The roughened surface serves as the hooks for receiving loops on straps 14. Rough features are also seen at 18 on the pads of FIG. 1.

Returning again to FIG. 1, the scrotal support member 16 is again shown. The support member 16 is dimensioned to support male organs when the patient is in a bed-ridden or otherwise recumbent state. The support member 16 may be of any shape, though a rectangular shape is believed to provide the best profile for appropriate support. As noted, the support member 16 may be fabricated from any material, though it preferably is fabricated from a soft, breathable material.

In the arrangement of FIG. 1, the skin pads 12 are separate from the straps 14. However, in one arrangement the skip pads 12 are integral with the straps 14.

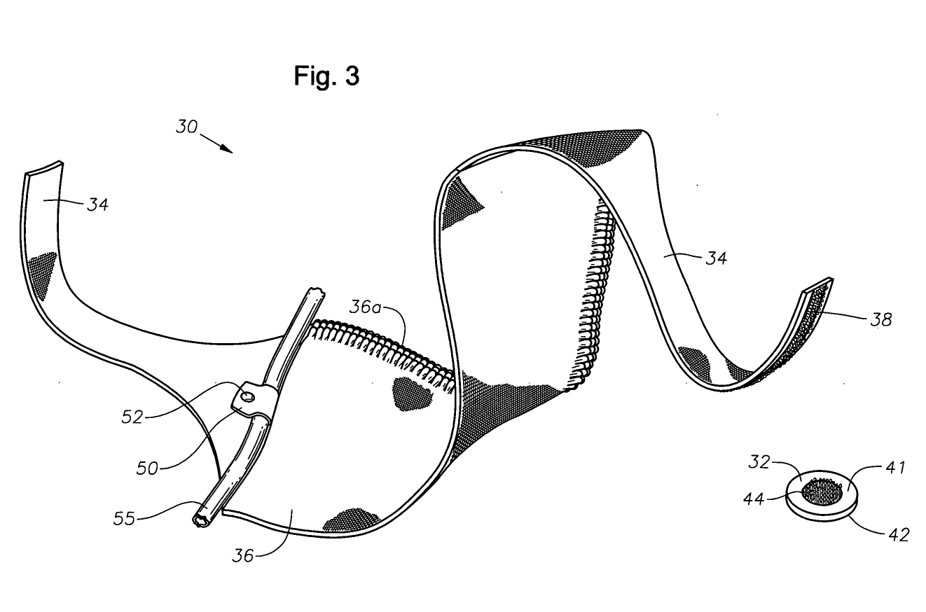

FIG. 3 presents a perspective view of a male support device 30, in an alternate embodiment. A scrotal support member is seen at 36. In this arrangement, the support member 36 includes an upper edge 36a that defines an elastic band. This provides an elastic and resilient nature to the male organ support device 30, and allows a single device 30 to better fit a multitude of patient sizes. The support device 30 employs one pair of straps 34 rather than two pairs of straps. The straps 34 are affixed to the support member 36 on opposing sides.

An alternate connection arrangement is provided for the skin pads in FIG. 3. A skin pad is seen at 32, exploded away from a strap 34. The skin pad 32 has a soft outer foam portion 41 and an inner attachment member 44. The inner attachment member 44 may be a snap or other connector. In the view of FIG. 3, the inner attachment member 44 is a loop material that receives an elongated hook material 38 along an outer surface of the strap 34. The elongated nature of the hook material 38 allows the health care provider or patient to adjust the tension of the support device 30 without moving the pad 32. Various configurations for the attachment point may be employed, including a square geometry that has a width that matches the width of the straps 34.

A method for supporting a male organ is also provided herein. In general, the method first comprises the step of providing a male organ support device as described above, in any embodiment. For purposes of example, the method will be described in connection with the embodiment of support device 30 presented in FIG. 3. However, it is understood that the support device could be the device 10 of FIG. 1, or any other embodiment that falls within the spirit and scope of the present inventions. Next, at least one pair of pads 32 is placed onto the body of the male patient. The adhesive pads 32 are preferably placed in such a manner as to secure the device in place without binding or causing tightness against the patient's skin. For example, the pads may be placed on the upper thigh regions of the patient. More specifically, the pads 32 may be placed in the high anterior thigh area. Alternatively, the pads 32 may be placed just above the pubic bone and below the waist line on opposite sides of the genitals, and outside the range of dense pubic hairs. Where two pairs of pads are employed, one pair may placed above the pubic bone, and another pair placed in the upper thigh area.

It will be understood by one of ordinary skill in the art in possession of this disclosure that a position of the pads 32 that is too superior to the scrotum might lead to the device 10 unduly pulling the perineum. A high position on the thigh, lateral and slightly superior to the scrotum, would help avoid such tightness. The scope of the present invention is not limited by the precise positioning of the pads.

Next, the scrotal support member 36 is positioned under the patient's scrotum. The scrotum is then raised into a comfortable position by lifting the opposing straps 34 by the free ends. The straps 34 are then affixed to the corresponding skin pads 32. Where two or more pairs of straps are employed, this latter step is repeated in order to affix the additional pairs of straps to corresponding pads. Each of the straps 34 is of adequate length to allow connection with a pad 32 and to be readily adjusted. Tension in the device 30 is adjusted, and the strap connector 38 is connected to the inner attachment member 44. Any excess length of a strap 34 can be trimmed with scissors if desired.

It can be seen that a waistband is not needed for supporting genitalia of a male patient. Further, a support device is provided that is comfortable, adjustable and disposable.

In a further alternate embodiment of a support device, the support device could be provided with a connection member for securing a catheter in place. In some male patients a Foley catheter is inserted through the urethra. The catheter extends into the bladder in order to drain urine. It is a constant nursing problem to keep the catheter from being pulled. In this respect, the drainage tubing is typically several feet in length—even up to five feet—and connects to a drainage bag at the side of the bed. The tubing can get caught in the bed sheets or on other objects such as bed railing. In some instances, the tubing may get caught below a patient's knee. When the patient straightens out his leg or when the patient is straightened up in bed, the tubing is pulled. In some instances, a nurse may think that the catheter has more slack than it does and end up pulling the tubing as against the urethra. Even the weight of the drainage tubing itself can be painful.

In order to militate against such circumstances, a connection member may be placed along the side of the support device to hold the catheter in place. The connection member may be a loop of Velcro™ or an adhesive material. Such a connection member decreases catheter movement and also helps prevent trauma to the meatus where the urethra ends.

The above described method may alternately include the step of securing a catheter to the support device. FIG. 3 shows a catheter connector 50 on an inner surface of the scrotal support member 36. The connector 50 is seen securing a catheter tube 55 relative to the support device 30. To this end, the catheter connector 50 is folded over the tube 55 and secured by a snap 52 or other securement means, such as Velcro™.

I claim:

1. A device for supporting male genitalia of a patient in a recumbent position, comprising:
a scrotal support member having first and second opposite ends;
at least one lateral support strap secured to each of the first and second ends; and
a plurality of pads corresponding to a respective lateral support strap, each pad having a first surface to be releasably secured to the patient, and a second surface connectible to the respective lateral support strap.

2. The device of claim 1, wherein the scrotal support member has two lateral support straps on each side.

3. The device of claim 1, wherein the scrotal support member has one lateral support strap on each side.

4. The device of claim 1, wherein each of the plurality of pads further comprises:
an adhesive material on the first surface.

5. The device of claim 4, wherein each of the plurality of pads is separate from the respective support strap so that the pad releasably receives a free end of a respective support strap.

6. The device of claim 4, wherein each of the plurality of pads is integral with a free end of a respective support strap.

7. The device of claim 5, wherein each of the plurality of pads further comprises:
an attachment member for connecting to a corresponding attachment feature on the respective lateral support strap.

8. The device of claim 7, wherein:
the attachment member on each of the plurality of pads and the corresponding attachment feature on the respective lateral support strap define a hook-and-loop attachment.

9. The device of claim 7, wherein:
the attachment member on each of the plurality of pads and the corresponding attachment feature on the respective lateral support strap define a snap attachment.

10. The device of claim 1, wherein the scrotal support member further comprises a connection member for securing a catheter in position.

11. A method for supporting male genitalia of a patient in a recumbent position, comprising the steps of:
providing a scrotal support member, the scrotal support member having first and second opposite ends with a lateral support strap secured to each of the first and second ends;
placing the scrotal support member under the patient's genitalia;
securing a first skin pad on the patient on one side of his body; and
securing a second skin pad on the patient on the other side of his body opposite his genitalia so as to support the genitalia of the patient.

12. The method of claim 11, wherein:
each of the plurality of pads is separate from the respective support strap so that the pad releasably receives a free end of a respective support strap; and
each of the skin pads comprises a first surface to be releasably secured to the skin of the patient, and a second surface for releasably receiving the respective lateral support strap.

13. The method of claim 12, further comprising the step of:
releasably securing each of the lateral support straps to a corresponding skin pad so as to support the genitalia of the patient.

14. The method of claim 13, further comprising the step of:
adjusting the tension in the support straps by moving the position of at least one of the support straps longitudinally relative to a corresponding skin pad; and
attaching the longitudinally adjusted support strap to the corresponding skin pad.

15. The method of claim 11, wherein:
each of the plurality of pads is integral with a free end of a respective support strap; and
the method further comprises the step of adjusting the tension in the support straps by moving the position of at least one of the support straps relative the patient.

16. The method of claim 11, wherein the scrotal support member has two lateral support straps on each side.

17. The method of claim 11, wherein each of the plurality of pads further comprises:
an adhesive material on a first surface; and
an attachment member for connecting to a corresponding attachment feature on the respective lateral support strap on a second surface.

18. The method of claim 12, wherein:
the attachment member on each of the plurality of pads and the corresponding attachment feature on the respective lateral support strap define a hook-and-loop attachment.

19. The method of claim 11, further comprising the step of securing a catheter to the scrotal support member.

20. A method for securing a catheter tube relative to a male patient, the catheter being inserted through the urethra of the patient, comprising the steps of:
providing a scrotal support member, the scrotal support member having first and second opposite ends with a lateral support strap secured to each of the first and second ends;
placing the scrotal support member under the patient's genitalia;
securing a first skin pad on the patient on one side of his body;
securing a second skin pad on the patient on the other side of his body opposite his genitalia so as to support the genitalia of the patient; and
connecting the catheter tube to the scrotal support member.

21. The method of claim 20, further comprising the step of:
releasably securing each of the lateral support straps to a corresponding skin pad so as to support the genitalia of the patient.
